(19) United States
(12) Patent Application Publication
(10) Pub. No.: US 2009/0214592 A1
(43) Pub. Date: Aug. 27, 2009

(54) Adjuvant composition comprising aluminium phosphate and 3D-MPL

(76) Inventor: Derek O'Hagan, Siena (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 11/884,610

(22) PCT Filed: Feb. 16, 2006
(86) PCT No.: PCT/GB06/00557

§ 371 Date: Nov. 12, 2008

Related U.S. Application Data
(60) Provisional application No. 60/653,741, filed on Feb. 16, 2005.

(51) Int. Cl. A61K 39/39; A61K 39/29

(52) U.S. Cl. 424/227.1; 424/283.1; 424/184.1

(57) ABSTRACT

An immunogenic composition comprising: (i) an antigen; (ii) an aluminum phosphate adjuvant; and (iii) a 3-O-deacylated monophosphoryl lipid A adjuvant. Components (ii) and (iii) can also be used as a separate adjuvant system. Various features of the compositions are disclosed, including that at least 50% of the 3-O-deacylated monophosphoryl lipid A adjuvant should be adsorbed to the aluminum phosphate adjuvant. The adjuvant mixture is particularly useful with hepatitis B virus surface antigen.

### TECHNICAL FIELD

This invention is in the field of vaccine adjuvants.

### BACKGROUND ART

Aluminum salts, often referred to generically as ‘alum’, are the classic vaccine adjuvant. Various further adjuvants have been described, and details can be found in texts such as references 1 and 2. One of these adjuvants is 3′ deacylated monophosphoryl lipid A (or ‘3D-MPL’).

References 3 to 10 report success in non-responder hepatitis patients using an adjuvant system referred to as ‘AS04’, said to include both 3D-MPL and alum [11-14. It is an object of the invention to provide modifications and improvements to this adjuvant system.

### DISCLOSURE OF THE INVENTION

Compositions of the invention include an aluminum phosphate adjuvant and a 3D-MPL adjuvant. This double adjuvant combination has already been described in general terms in references 12-14, but the invention discloses a number of modifications of the combination:
(a) the composition should have an osmolality of between 200 and 400 mOsm/kg.
(b) the composition should have a pH between 5 and 7.5.
(c) the composition should be buffered.
(d) at least 50% of the 3D-MPL in the vaccine should be adsorbed to aluminum phosphate.
(e) the 3D-MPL in the vaccine should take the form of micellar structures with a diameter of less than 150 nm.
(f) the 3D-MPL in the vaccine should be a mixture of different acylated forms, preferably including at least 10% of the 6-acyl-chain form.
(g) the composition may include one or more of: polyoxyethylene sorbitan monooleate; sorbitol; triethanolamine; a triethylammonium ion; lactose; sucrose; trehalose; mannitol.
   These modifications can used independently or in combination.

Thus the invention provides an adjuvant composition comprising: (i) an aluminum phosphate adjuvant; and (ii) a 3-O-deacylated monophosphoryl lipid A adjuvant, characterised in that the composition has an osmolality of between 200 and 400 mOsm/kg.

The invention also provides an adjuvant composition comprising: (i) an aluminum phosphate adjuvant; and (ii) a 3-O-deacylated monophosphoryl lipid A adjuvant, characterised in that the composition has a pH of between 5 and 7.5.

The invention also provides an adjuvant composition comprising: (i) an aluminum phosphate adjuvant; and (ii) a 3-O-deacylated monophosphoryl lipid A adjuvant, characterised in that the composition is buffered e.g. to a pH of between 5 and 7.5.

The invention also provides an adjuvant composition comprising: (i) an aluminum phosphate adjuvant; and (ii) a 3-O-deacylated monophosphoryl lipid A adjuvant, characterised in that at least 50% of the 3-O-deacylated monophosphoryl lipid A is adsorbed to the aluminum phosphate.

The invention also provides an adjuvant composition comprising: (i) an aluminum phosphate adjuvant; and (ii) a 3-O-deacylated monophosphoryl lipid A adjuvant, characterised in that the composition has less than 50 μg/ml of unadsorbed 3-O-deacylated monophosphoryl lipid A.

The invention also provides an adjuvant composition comprising: (i) an aluminum phosphate adjuvant; and (ii) a 3-O-deacylated monophosphoryl lipid A adjuvant, characterised in that the 3-O-deacylated monophosphoryl lipid A adjuvant is in the form of particles having a diameter of less than 150 nm.

The invention also provides an adjuvant composition comprising: (i) an aluminum phosphate adjuvant; and (ii) a 3-O-deacylated monophosphoryl lipid A adjuvant comprising a mixture of acylated disaccharides, wherein each disaccharide: (a) has two β-1′,6-linked 2-deoxy-2-aminoglucose monosaccharide subunits; (b) is phosphorylated at the 4′ position; (c) is unsubstituted at the 1, 3 and 6′ positions, (d) is O-acylated at the 3′ position, and (e) is N-acylated at the 2 and 2′ positions, and wherein the mixture of acylated disaccharides includes at least 10% by weight of a component in which each of the acyl groups at the 2, 2′ and 3′ positions is itself substituted at an/aliphatic carbon atom with an O-acyl group.

The invention also provides an adjuvant composition comprising: (i) an aluminum phosphate adjuvant; (ii) a 3-O-deacylated monophosphoryl lipid A adjuvant; and at least one substance selected from the group consisting of: sorbitol; triethanolamine; a triethylammonium ions lactose; sucrose; trehalose; and mannitol.

These various features may be used in combination. Thus the invention provides an adjuvant composition comprising: (i) an aluminum phosphate adjuvant; and (ii) a 3-O-deacylated monophosphoryl lipid A adjuvant, characterised in that the composition has one or more of the following properties:
(1) an osmolality between 200 and 400 mOsm/kg;
(2) a pH of between 5 and 7.5;
(3) it comprises a buffer;
(4) at least 50% of the 3-O-deacylated monophosphoryl lipid A is adsorbed to the aluminum phosphate;
(5) it has less than 50 μg/ml of unadsorbed 3-O-deacylated monophosphoryl lipid A;
(6) the 3-O-deacylated monophosphoryl lipid A adjuvant is in the form of particles having a diameter of less than 150 nm;
(7) the 3-O-deacylated monophosphoryl lipid A adjuvant comprises a mixture of acylated disaccharides, wherein each disaccharide: (a) has two β-1′,6-linked 2-deoxy-2-aminoglucose monosaccharide subunits; (b) is phosphorylated at the 4′ position; (c) is unsubstituted at the 1, 3 and 6′ positions, (d) is O-acylated at the 3′ position, and (e) is N-acylated at the 2 and 2′ positions, and wherein the mixture of acylated disaccharides includes at least 10% by weight of a component in which each of the acyl groups at the 2, 2′ and 3′ positions is itself substituted at an aliphatic carbon atom with an O-acyl group; and/or
(8) it comprises at least one substance selected from the group consisting of: sorbitol; triethanolamine; a triethylammonium ion; lactose; sucrose; trehalose; and mannitol.

The invention also provides an immunogenic composition comprising an adjuvant composition of the invention, and further comprising (iii) an antigen.

### The Aluminum Phosphate Adjuvant

Compositions of the invention include an aluminum phosphate adjuvant and a 3D-MPL adjuvant.

The term “aluminum phosphate” is conventional in the field, but is not a precise description of the actual chemical compound which is present [e.g. see chapter 9 of reference 2]. The invention can use any of the “aluminum phosphate” adjuvants that are in general use as adjuvants, which are typically aluminum hydroxyphosphates, often also containing a small amount of sulfate (i.e. aluminum hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls [chapter 9 of ref. 2].

The aluminum salt can take any suitable physical form, but will typically be amorphous.

The PO₄/Al³⁺ molar ratio of an aluminum phosphate adjuvant will generally be between 0.3 and 1.2, preferably between 0.8 and 1.2, and more preferably 0.95±0.1. The aluminum phosphate will generally be amorphous, particularly for hydroxyphosphate salts. A typical adjuvant is amorphous aluminum hydroxyphosphate with PO₄/Al molar ratio between 0.84 and 0.92, included at 0.6 mg Al³⁺/ml. The aluminum phosphate will generally be particulate. Typical diameters of the particles are in the range 0.5-20 μm (e.g. about 5-10 μm) after any antigen and/or 3D-MPL adsorption.

The PZC of aluminum phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminum phosphates used according to the invention will generally have a PZC of between 4.0 and 7.0, more preferably between 5.0 and 6.5 e.g. about 5.7.

The aluminum phosphate is preferably used in the form of an aqueous solution to which 3D-MPL (and, optionally, an antigen) is added (NB: it is standard to refer to aqueous aluminum phosphate as a “solution” although, on a strict physicochemical view, the salt is insoluble and forms a suspension). It is preferred to dilute the aluminum phosphate to the required concentration and to ensure a homogenous solution before the addition of the 3D-MPL and/or the antigen.

The concentration of Al³⁺ prior to addition of 3D-MPL and/or antigen is generally between 0 and 10 mg/ml. A preferred concentration is between 0.5 and 3 mg/ml.

An aluminum phosphate solution used to prepare a composition of the invention may contain a buffer (e.g. a phosphate or a histidine or a Tris buffer), but this is not always necessary. The aluminum phosphate solution is preferably sterile and pyrogen-free. The aluminum phosphate solution may include free aqueous phosphate ions e.g. present at a concentration between 1.0 and 20 mM, preferably between 5 and 15 mM, and more preferably about 10 mM. The aluminum phosphate solution may also comprise sodium chloride. The concentration of sodium chloride is preferably in the range of 0.1 to 100 mg/ml (e.g. 0.5-50 mg/ml, 1-20 mg/ml, 2-10 mg/ml) and is more preferably about 3±1 mg/ml. The presence of NaCl facilitates the correct measurement of pH prior to adsorption of other components, and also affects osmolality.

### The 3D-MPL Adjuvant

Compositions of the invention include an aluminum phosphate adjuvant and a 3D-MPL adjuvant.

3-O-deacylated monophosphoryl lipid A (3D-MPL) has also been referred to as 3 de-O-acylated monophosphoryl lipid A or as 3-O-desacyl-4′-monophosphoryl lipid A. The name indicates that position 3 of the reducing end glucosamine in monophosplhoryl lipid A is de-acylated. It has been prepared from a heptoseless mutant of Salmonella minnesota, and is chemically similar to lipid A but lacks an acid-labile phosphoryl group and a base-labile acyl group. It activates cells of the monocyte/macrophage lineage and stimulates release of several cytokines, including IL-1, IL-12, TNF-α and GM-CSF. Preparation of 3D-MPL was originally described in reference 15, and the product has been manufactured and sold by Corixa Corporation under the trade name MPL™. Further details can be found in references 16 to 19.

Typical compositions include 3D-MPL at a concentration of between 25 μg/ml and 200 μg/ml e.g. in the range 50-150 μg/ml, 75-125 μg/ml, 90-110 μg/ml, or about 100 μg/ml. It is usual to administer between 25-75 μg of 3D-MPL per dose e.g. between 45-55 μg, or about 50 μg 3D-MPL per dose.

Advantageously, the 3D-MPL is adsorbed onto the aluminum phosphate. Preferably at least 50% (by weight) of the 3D-MPL is adsorbed e.g. ≧60%, ≧70%, ≧80%, ≧90%, ≧95%, ≧98% or more. The percentage that is adsorbed can be measured in the same way as for antigens (see below). In a composition having a total 3D-MPL concentration of 100 μg/ml then the concentration of unadsorbed 3D-MPL should be less than 50 μg/ml e.g. ≦40 μg/ml, ≦35 μg/ml, ≦30 μg/ml, ≦25 μg/ml, ≦20 μg/ml, ≦15 μg/ml, ≦10 μg/ml, ≦5 μg/ml, ≦2 μg/ml, ≦1 μg/ml, etc.

3D-MPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position. The group attached to carbon 2 has formula —NH—CO—CH₂—CR¹R^{1″}. The group attached to carbon 2′ has formula —NH—CO—CH₂—CR²R^{2′}. The group attached to carbon 3′ has formula —O—CO—CH₂—CR³R^{3′}. A representative structure is:

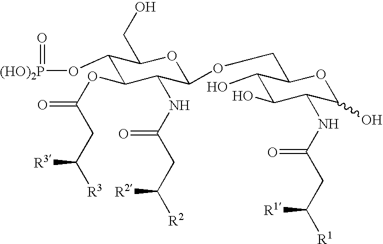

Groups R¹, R² and R³ are each independently —(CH₂)ₙ—CH₃. The value of it is preferably between 8 and 16, more preferably between 9 and 12, and is most preferably 10.

Groups R^{1′}, R^{2′} and R^{3′} can each independently be: (a) —H; (b) —OH; or (c) —O—CO—R⁴, where R⁴ is either —H or —(CH₂)ₘCH₃, wherein the value of m is preferably between 8 and 16, and is more preferably 10, 12 or 14. At the 2 position, m is preferably 14. At the 2′ position, m is preferably 10. At the 3′ position, m is preferably 12. Groups R^{1′}, R^{2′} and R^{3′} are thus preferably —O-acyl groups from dodecanoic acid, tetradecanoic acid or hexadecanoic acid.

When all of R^{1′}, R^{2′} and R^{3′} are —H then the 3D-MPL has only 3 acyl chains (one on each of positions 2, 2′ and 3′). When only two of R^{1′}, R^{2′} and R^{3′} are —H then the 3D-MPL can have 4 acyl chains. When only one of R^{1′}, R^{2′} and R^{3′} is —H then the 3D-MPL can have 5 acyl chains. When none of R^{1′}, R^{2′} and R^{3′} is —H then the 3D-MPL can have 6 acyl chains. The 3D-MPL adjuvant used according to the invention can be a mixture of these forms, with from 3 to 6 acyl chains, but it is preferred to include 3D-MPL with 6 acyl chains in the mixture, and in particular to ensure that the 6 acyl chain form makes up at least 10% by weight of the total 3D-MPL e.g. ≧20%, ≧30%, ≧40%, ≧50% or more. 3D-MPL with 6 acyl chains has been found to be the most adjuvant-active form.

Thus the most preferred form of 3D-MPL for inclusion in compositions of the invention is:

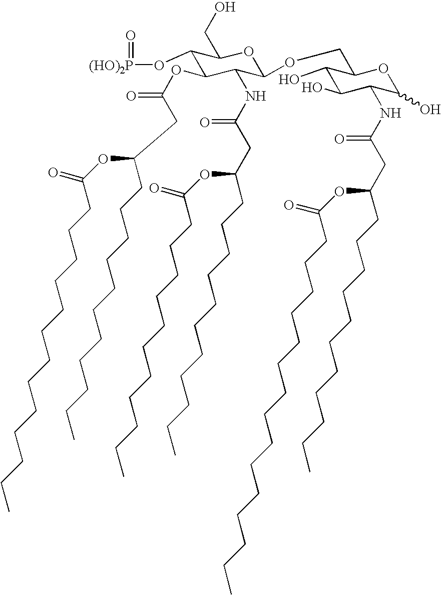

Where 3D-MPL is used in the form of a mixture then references to amounts or concentrations of 3D-MPL in compositions of the invention refer to the combined 3D-MPL species in the mixture.

In aqueous conditions, 3D-MPL can form micellar aggregates or particles with different sizes e.g. with a diameter <150 nm or >500-nm. Either or both of these can be used with the invention, and the better particles can be selected by routine assay. Smaller particles (e.g. small enough to give a clear aqueous suspension of 3D-MPL) are preferred for use according to the invention because of their superior activity [20]. Preferred particles have a mean diameter less than 150 nm, more preferably less than 120 nm, and can even have a mean diameter less than 100 nm. In most cases, however, the mean diameter will not be lower than 50 nm.

Where 3D-MPL is adsorbed to aluminum phosphate then it may not be possible to measure the 3D-MPL particle size directly, but particle size can be measured before adsorption takes place.

Particle diameter can be assessed by the routine technique of dynamic light scattering, which reveals a mean particle diameter. Where a particle is said to have a diameter of x nm, there will generally be a distribution of particles about this mean, but at least 50% by number (e.g. ≧60%, ≧70%, ≧80%, ≧90%, or more) of the particles will have a diameter within the range x±25%.

### The Optional Antigen

The adjuvant system of the invention is preferably used in combination with an antigen in order to enhance immune responses that result from administration of the antigen.

Preferred antigens for use with the adjuvant system of the invention are viral antigens, such as those from hepatitis B virus (HBV), human papillomavirus (HPV) or herpes simplex virus (HSV). The adjuvant system is also suitable for use with parasite antigens, such as those from Plasmodium falciparum.

An antigen concentration of between 5 μg/ml and 50 μg/ml is typical e.g. between 10-30 μg/ml, between 15-25 μg/ml, or about 20 μg/ml. An amount of antigen per dose of between 5 μg/dose and 50 μg/dose is also typical e.g. between 10-30 μg/dose, between 15-25 μg/dose, or about 20 μg/dose.

The antigen is preferably adsorbed to the aluminum phosphate adjuvant. The percentage of a particular antigen in a composition that is adsorbed is preferably at least 50% (by weight) e.g. ≧60%, ≧70%, ≧80%, ≧90%, ≧95%, ≧98% or higher e.g. up to 100%. The percentage of an antigen in a composition that is adsorbed can conveniently be measured by separating the adsorbed material from the non-adsorbed material e.g. by centrifugation, in which aluminum-adsorbed antigen will readily form a pellet, whereas the unadsorbed antigen will remain in the supernatant. The amount of antigen in the supernatant (e.g. measured by ELISA) can be subtracted from the total amount of that antigen in the composition, and then the adsorbed percentage can be calculated. It is preferred that the antigen is totally adsorbed i.e. none is detectable in supernatant.

Hepatitis B virus (HBV) is one of the known agents which causes viral hepatitis. The HBV virion consists of an inner core surrounded by an outer protein coat or capsid, and the viral core contains the viral DNA genome. The major component of the capsid is a protein known as HBV surface antigen or, more commonly, ‘HBsAg’, a 226-amino acid polypeptide with a molecular weight of ˜24 kDa. All existing hepatitis B vaccines contain HBsAg, and when this antigen is administered to a normal vaccine it stimulates the production of anti-HBsAg antibodies which protect against HBV infection.

Thus the preferred HBV antigen is HBsAg. HBsAg can be adsorbed onto aluminum phosphate using the methods described in ref. 21. Adsorption to aluminum phosphate contrasts with the well-known ENGERIX-B™ product (where HBsAg is adsorbed to aluminum hydroxide), but is the same as in the HEPACCINE™ and RECOMBIVAX™ products. As mentioned in reference 22, aluminum phosphate can be a better adjuvant for HBsAg than aluminum hydroxide.

For vaccine manufacture, HBsAg can be made in two ways. The first method involves purifying the antigen in particulate form from the plasma of chronic hepatitis B carriers, as large quantities of HBsAg are synthesized in the liver and released into the blood stream during an HBV infection. The second way involves expressing the protein by recombinant DNA methods. HBsAg for use with the present invention may be prepared in either way, but it is preferred to use HBsAg which has been recombinantly expressed. In particular, it is preferred that the HBsAg is prepared by expression in a Saccharomyces cerevisiae yeast. Unlike native HBsAg (i.e. as in the plasma-purified product), yeast-expressed HBsAg is generally non-glycosylated, and this is the most preferred form of HBsAg for use with the invention, because it is highly immunogenic and can be prepared without the risk of blood product contamination. The yeast-expressed HBsAg is advantageously in the form of substantially-spherical particles (average diameter of about 20 nm), including a lipid matrix comprising phospholipids.

After purification HBsAg may be subjected to dialysis (e.g. with cysteine), which can be used to remove any mercurial preservatives such as thimerosal that may have been used during HBsAg preparation [23].

In addition to the ‘S’ sequence, a surface antigen may include all or part of a pre-S sequence, such as all or part of a pre-S1 and/or pre-S2 sequence.

A preferred HPV antigen for use with the invention is the L1 capsid protein, which can assemble to form structures known as virus-like particles (VLPs). The VLPs can be produced by recombinant expression of L1 in yeast cells (e.g. in S. cerevisiae) or in insect cells (e.g. in Spodoptera cells, such as S. frugiperda, or in Drosophila cells). For yeast cells, plasmid vectors can carry the L1 gene(s); for insect cells, baculovirus vectors can carry the L1 gene(s). More preferably, the composition includes L1 VLPs from both HPV-16 and HPV-18 strains. This bivalent combination has been shown to be highly effective [24]. In addition to HPV-16 and HPV-18 strains, it is also possible to include L1 VLPs from HPV-6 and HPV-11 strains. The use of oncogenic HPV strains is also possible. A vaccine may include between 20-60 μg/ml (e.g. about 40 μg/ml) of L1 per HPV strain.

A preferred HSV antigen for use with the invention is membrane glycoprotein gD. It is preferred to use gD from a HSV-2 strain (‘gD2’ antigen). The composition can use a form of gD in which the C-terminal membrane anchor region has been deleted [25] e.g. a truncated gD comprising amino acids 1-306 of the natural protein with the addition of aparagine and glutamine at the C-terminus. This form of the protein includes the signal peptide which is cleaved to yield a mature 283 amino acid protein. Deletion of the anchor allows the protein to be prepared in soluble form.

A preferred P. falciparum antigen for use with the invention is based on the circumsporozoite (CS) protein. This can take the form of a recombinant protein that fuses a part of the CS protein with HBsAg, known as ‘RTS,S’, or TRAP. RTS is a hybrid protein comprising substantially all the C-terminal portion of CS linked via four amino acids of the preS2 portion of HBV surface antigen to HBsAg [26]. When expressed in yeast (particularly in S. cerevisiae) RTS is produced as a lipoprotein particle (including in particular phospholipids), and when it is co-expressed with the S antigen from HBV it produces a mixed particle known as RTS,S. A RTS:S ratio of about 1:4 is useful. TRAP antigens are described in reference 27.

### Pharmaceutical Compositions

In addition to the adjuvant and antigen components, compositions of the invention may include further components. These components may have various sources. For example, they may be present in one of the antigen or adjuvant components that is used during manufacture or may be added separately from the antigenic components.

Preferred compositions of the invention include one or more pharmaceutical carrier(s) and/or excipient(s).

To control tonicity, it is preferred to include a physiological salt, such as a mineral salt e.g. a sodium salt. Sodium chloride (NaCl) is preferred, which may be present at between 1 and 20 mg/ml. This can be present during the mixing of the adjuvants and during the mixing of antigen with the adjuvant(s).

Compositions will generally have an osmolality of between 200 mOsm/kg and 400 mOsm/kg, preferably between 240-360 mOsm/kg, and will more preferably fall within the range of 290-300 mOsm/kg. Osmolality has previously been reported not to have an impact on pain caused by vaccination [28], but keeping osmolality in this range is nevertheless preferred.

Compositions of the invention may include one or more buffers. Typical buffers include: a phosphate buffer; a Tris buffer; a borate buffer; a succinate buffer; a histidine buffer; or a citrate buffer. To avoid competition between phosphate groups in the buffer and in the 3D-MPL then buffers other than phosphate buffer may be preferred. Buffers will typically be included in the 5-20 mM range.

The pH of a composition of the invention will generally be between 5.0 and 7.5, and more typically between 5.0 and 6.0 for optimum stability, or between 6.0 and 7.0.

Due to the adsorbed nature of antigens, final vaccine products may be a suspension with a cloudy appearance. This appearance means that microbial contamination is not readily visible, and so the vaccine preferably contains an antimicrobial agent. This is particularly important when the vaccine is packaged in multidose containers. Preferred antimicrobials for inclusion are 2-phenoxyethanol and thimerosal. It is preferred, however, not to use mercurial preservatives (e.g. thimerosal) during the process of the invention. However, the presence of trace amounts may be unavoidable if antigen was treated with such a preservative before being used to prepare the composition of the invention. For safety, however, it is preferred that the final composition contains less than about 25 ng/ml mercury. More preferably, the final vaccine product contains no detectable thimerosal. This will generally be achieved by removing the mercurial preservative from an antigen preparation prior to its addition in the process of the invention or by avoiding the use of thimerosal during the preparation of the components used to make the composition.

During manufacture, dilution of components to give desired final concentrations will usually be performed with WFI (water for injection).

The concentration of aluminum phosphate in a composition of the invention, expressed in terms of Al³⁺, is preferably less than 5 mg/ml e.g. ≦4 mg/ml, ≦3 mg/ml, ≦2 mg/ml, ≦1 mg/ml, etc.

The concentration of 3D-MPL in a composition of the invention is preferably less than 200 μg/ml e.g. ≦150 μg/ml, ≦125 μg/ml, ≦110 μg/ml, ≦100 μg/ml, etc.

The concentration of an individual antigen in a composition of the invention is preferably less than 60 μg/ml e.g. ≦55 μg/ml, ≦50 μg/ml, ≦45 μg/ml, ≦40 μg/ml, etc.

Compositions of the invention are preferably administered to patients in 0.5 ml doses. References to 0.5 ml doses will be understood to include normal variation e.g. 0.5 ml±0.1 ml, 0.5 ml±0.05 ml, etc.

Preferred compositions have about 50 kg 3D-MPL and about 0.5 mg aluminum adjuvant per dose.

The invention can provide bulk material which is suitable for packaging into individual doses, which can then be distributed for administration to patients. Concentrations mentioned above are typically concentrations in final packaged dose, and so concentrations in bulk vaccine may be higher (e.g. to be reduced to final concentrations by dilution).

Compositions of the invention will generally be in aqueous form.

Further components that may be present in the compositions of the invention include: polyoxyethylene sorbitan monooleate (‘Tween 80’), which may have been used to prevent 3D-MPL aggregation [20]; sorbitol, which may also have been used to prevent 3D-MPL aggregation; triethanolarmine, which may have been used to solubilise the 3D-MPL; a triethylammonium ion, which may also have been used to solubilise the 3D-MPL; lactose; sucrose; trehalose; and/or mannitol.

### Processes of the Invention

The invention provides a process for manufacturing an adjuvant composition of the invention, comprising the step of combining: (i) an aluminum phosphate adjuvant; and (ii) a 3-O-deacylated monophosphoryl lipid A adjuvant.

The invention also provides a process for manufacturing a composition of the invention, comprising the step of combining: (i) an antigen; (ii) an aluminum phosphate adjuvant; and (iii) a 3-O-deacylated monophosphoryl lipid A adjuvant. The components (i), (ii) and (iii) can be combined in any order, but the antigen and aluminum phosphate are preferably mixed first, and then the 3D-MPL is added to the antigen/aluminum phosphate mixture. As an alternative, the 3D-MPL and aluminum phosphate are mixed first, and then the antigen is added to the adjuvant mixture.

The invention provides a process for manufacturing a composition of the invention, comprising the steps of: (a) expressing an antigen in a recombinant host; (b) purifying the antigen; and (c) combining the purified antigen with (i) an aluminum phosphate adjuvant and (ii) a 3-O-deacylated monophosphoryl lipid A adjuvant. The three components combined in step (c) can be combined in any order, as described above. Preferred recombinant hosts are yeasts and insect cells, as described above.

The invention provides a process for manufacturing a composition of the invention, comprising the steps of: (a) combining an antigen, an aluminum phosphate adjuvant and a 3-O-deacylated monophosphoryl lipid A adjuvant; (b) measuring the osmolality of the composition; and, if the osmolality is outside the range of 200-400 mOsm/kg, (c) adjusting the osmolality to fall within the range of 200-400 mOsm/kg. The adjustment may involve the addition of a physiological salt, such as a sodium salt e.g. sodium chloride.

The invention provides a process for manufacturing a composition of the invention, comprising the steps of: (a) combining an antigen, an aluminum phosphate adjuvant and a 3-O-deacylated monophosphoryl lipid A adjuvant; (b) measuring the pH of the composition; and, if the pH is outside the range of 5.0 to 7.5, (c) adjusting the pH to fall within the range of 5.0 to 7.5. The adjustment may involve the addition of an acid or a base.

The invention provides a process for manufacturing a composition of the invention, comprising the step combining (i) an antigen, (ii) an aluminum phosphate adjuvant and (iii) a 3-O-deacylated monophosphoryl lipid A adjuvant, wherein the 3-O-deacylated monophosphoryl lipid A in component (iii) is in the form of particles having a diameter of less than 150 nm. Components (i), (ii) and (iii) may be mixed in any order. Component (iii) may additionally comprise polyoxyethylene sorbitan monooleate and/or sorbitol.

After combining the antigen and the adjuvants, the processes of the invention may comprise a step of extracting and packaging a 0.5 ml sample of the mixture into a container. For multidose situations, multiple dose amounts will be extracted and packaged together in a single container.

The processes of the invention may comprise the further step of packaging the vaccine into containers for use. Suitable containers include vials and disposable syringes (preferably sterile ones).

### Packaging Compositions of the Invention

Where a composition of the invention is packaged into vials, these are preferably made of a glass or plastic material. The vial is preferably sterilized before the composition is added to it. To avoid problems with latex-sensitive patients, vials are preferably sealed with a latex-free stopper. The vial may include a single dose of vaccine, or it may include more than one dose (a ‘multidose’ vial) e.g. 10 doses. When using a multidose vial, each dose should be withdrawn with a sterile needle and syringe under strict aseptic conditions, taking care to avoid contaminating the vial contents. Preferred vials are made of colorless glass.

Where the composition is packaged into a syringe, the syringe will not normally have a needle attached to it, although a separate needle may be supplied with the syringe for assembly and use. Safety needles are preferred. 1-inch 23-gauge, 1-inch 25-gauge and ⅝-inch 25-gauge needles are typical. Syringes may be provided with peel-off labels on which the lot number and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of butyl rubber. If the syringe and needle are packaged separately then the needle is preferably fitted with a butyl rubber shield. Grey butyl rubber is preferred. Preferred syringes are those marketed under the trade name “Tip-Lok”™.

Packaging into syringes is preferred, such that a physician or patient receives a pre-filled syringe.

Where a glass container (e.g. a syringe or a vial) is used, then it is preferred to use a container made from a borosilicate glass rather than from a soda lime glass.

After a composition is packaged into a container, the container can then be enclosed within a box for distribution e.g. inside a cardboard box, and the box will be labeled with details of the vaccine e.g. its trade name, a list of the antigens in the vaccine (e.g. ‘hepatitis B recombinant’, etc.), the presentation container (e.g. ‘Disposable Prefilled Tip-Lok Syringes’ or ‘10×0.5 ml Single-Dose Vials’), its dose (e.g. ‘each containing one 0.5 ml dose’), warnings (e.g. ‘For Adult Use Only’), an expiration date, an indication, etc. Each box might contain more than one packaged vaccine e.g. five or ten packaged vaccines (particularly for vials). If the vaccine is contained in a syringe then the package may show a picture of the syringe.

The vaccine may be packaged together (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

The packaged vaccine materials are preferably sterile.

The packaged vaccine materials are preferably non-pyrogenic e.g. containing <1 EU (endotoxin unit, a standard measure) per dose, and preferably <0.1 EU per dose.

The packaged vaccine materials are preferably gluten free.

The pH of any aqueous packaged vaccine materials is preferably between 5 and 8 e.g. between 5.5 and 6.5. The process of the invention may therefore include a step of adjusting the pH of the bulk vaccine prior to packaging.

The packaged vaccine is preferably stored at between 2° C. and 8° C. It should not be frozen.

### Methods of Treatment and Administration of the Vaccine

Compositions of the invention are suitable for administration to human patients, and the invention provides a method of raising an immune response in a patient, comprising the step of administering a composition of the invention to the patient.

The invention also provides a composition of the invention for use in medicine.

The invention also provides the use of (i) an antigen; (ii) an aluminum phosphate adjuvant; and (iii) a 3-O-deacylated monophosphoryl lipid A adjuvant, in the manufacture of a medicament for administering to a patient.

The methods and uses of the invention are particularly suitable for eliciting immune responses after being administered to patients, for protecting against and/or treating: HBV infection; HSV infection; genital herpes caused by HSV; HPV infection; genital warts caused by HPV; cervical cancer caused by HPV; and/or malaria.

Immunogenic compositions of the invention are preferably vaccines, for use in the prevention and/or treatment of infection.

In order to have full efficacy, a typical immunisation schedule may involve administering more than one dose. For example, doses may be at: 0 & 6 months (time 0 being the first dose); at 0, 1, 2 & 6 months; at day 0, day 21 and then a third dose between 6 & 12 months; or at 0, 1, 2, 6 & 12 months.

Compositions of the invention can be administered by intramuscular injection e.g. into the arm or leg

As compositions of the invention include an aluminum-based adjuvant, settling of components may occur during storage. The composition should therefore be shaken prior to administration to a patient. The shaken composition will be a turbid white suspension.

### Further Antigenic Components

As well as including HBsAg, HPV L1, HSV gD and/or a malaria antigen, compositions of the invention may include one or more further antigens. For instance, they may include one or more of: a hepatitis A virus antigen; a diphtheria toxoid; a tetanus toxoid; an inactivated poliovirus antigen; a cellular pertussis antigen; an acellular pertussis antigen, comprising a detoxified pertussis toxin, filamentous haemagglutinin and, optionally, the 69 kDa antigen; a conjugated H. influenzae type B capsular saccharide, typically with a tetanus toxoid as the carrier protein; a conjugated serogroup A N. meningitidis capsular saccharide; a conjugated serogroup C N. meningitidis capsular saccharide; a conjugated serogroup Y N. meningitidis capsular saccharide; a conjugated serogroup W135 N. meningitidis capsular saccharide; a conjugated S. pneumoniae capsular saccharide.

### Alternative to Aluminium Phosphate

For some applications, it may be useful to replace an aluminium phosphate adjuvant with an aluminium hydroxide adjuvant, or to combine aluminium hydroxide and phosphate adjuvants. In HPV and HSV vaccines, for instance, aluminium hydroxide may be preferable to aluminium phosphate. The above definitions of the invention can be amended accordingly.

The term “aluminum hydroxide” is conventional in the field, but is not a precise description of the actual chemical compound which is present [e.g. see chapter 9 of reference 2]. The invention can use any of the “aluminum hydroxide” adjuvants that are in general use as adjuvants, which are typically aluminum oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm¹ and a strong shoulder at 3090-3100 cm⁻¹ [chapter 9 of ref 2]. The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

It will be appreciated that ionisable groups may exist in the neutral form shown in formulae herein, or may exist in charged form e.g. depending on pH. Thus a phosphate group may be shown as —P—O—(OH)₂, this formula is merely representative of the neutral phosphate group, and other charged forms are encompassed by the invention. Similarly, sugar rings can exist in open and closed form and, while closed forms are shown in structural formulae herein, open forms are also encompassed by the invention.

### MODES FOR CARRYING OUT THE INVENTION

HBsAg expressed in recombinant S. cerevisiae is purified by a process involving cell recovery, precipitation, ultrafiltration, gel permeation, ion exchange, ultracentrifugation and desalting. The purified antigen is non-glycosylated and can be seen in the form of substantially-spherical particles (average diameter ˜20 nm).

The antigen is kept in a phosphate buffer solution and is adsorbed to an amorphous aluminum phosphate adjuvant (between 3-6 mg/ml Al⁺⁺⁺) for one hour at room temperature under agitation. The mixture is stored at room temperature for two weeks and then kept in a refrigerator. 3D-MPL adjuvant from Corixa is then added, allowed to adsorb onto the aluminium phosphate adjuvant, and any necessary dilution to a desired final antigen concentration is achieved using water for injection and sterile saline. This bulk vaccine is then packaged into individual doses in disposable syringes.

Vaccine manufactured in this way is initially tested in healthy adolescents and adults. The vaccine elicits a stronger immune response (seroprotection rates up to 100%, higher GMT values) than the ENGERIX B™ product among all age groups.

With an aluminium phosphate/3dMPL adjuvant mixture, seroprotection rates of 98.6% are seen when the vaccine is administered as a two-dose schedule (0 and 6 months), which is better than the 96.8% seen using ENGERIX B™ at 0, 1 and 6 months. GMTs are around 7800 (vs. 3700 with ENGERIX B™). After initial testing, testing moves to pre-hemodialysis patients and those already undergoing hemodialysis, aged 15 or older (mean age 58). These patients are HBV naïve. Single doses of this vaccine (20 μg HBsAg) are compared to double doses of ENGERIX B™, administered at 0, 1, 2 and 6 months. Seroprotection rates (%) and anti-HBsAg GMTs (mIU/ml) are as follows:

| | Adjuvant(s) | Time after first immunisation (months) | | | | | |
|---|---|---|---|---|---|---|---|
| | in vaccine | 2 | 6 | 7 | 12 | 24 | 30 |
| SP | AP + 3DMPL | 49 | 82 | 91 | 86 | 86 | 70 |
| (%) | AH | 22 | 66 | 84 | 77 | 77 | 53 |
| GMT | AP + 3DMPL | 80 | 250 | 3560 | 910 | 350 | 180 |
| (mIU/ml) | AH | 60 | 90 | 930 | 320 | 210 | 100 |

Thus these vaccines consistently raise better immune responses in hemodialysis adults than the market-leading ENGERIX B™ vaccine. Moreover, the onset of protection is more rapid (e.g. 75% of patients seroprotected at month 3 vs. 52% with ENGERIX B™, p<0.005) and persists for longer.

A further trial in HBV-naïve patients awaiting liver transplants reveals similar results. Vaccines are administered at day 0 and day 21 (plus a day 7 dose for ENGERIX B™), and then a final dose at between 6 and 12 months:

Measured after

| | Adjuvant(s) | Day 28 | Final dose |
|---|---|---|---|
| SP | AP + 3DMPL | 32 | 60 |
| (%) | AH | 21 | 32 |
| GMT | AP + 3DMPL | 20 | 480 |
| (mIU/ml) | AH | 40 | 280 |

The seroprotection rate is higher using the aluminium phosphate/3dMPL mixture (60% vs. 32%, p<0.035).

Satisfactory safety and reactogenicity is seen in all patients. Transient local discomfort is higher with the vaccines of the invention, but this resolves quickly and is an acceptable side effect when compared to the therapeutic benefit.

It will be understood that the invention has been described by way of example only and modifications may be made whilst remaining within the scope and spirit of the invention.

### REFERENCES

### The Contents of which are Hereby Incorporated by Reference

[1] Vaccine Adjuvants: Preparation Methods and Research Protocols (Volume 42 of Methods in Molecular Medicine series). ISBN: 1-59259-083-7. Ed. O'Hagan.
[2] Vaccine Design: The Subunit and Adjuvant Approach (eds. Powell & Newman) Plenum Press 1995 (ISBN 0-30644867-X).
[3] Desombere et al. (2002) Vaccine 20:2597-602.
[4] Levie et al. (2002) Scand J Infect Dis 34:610-4.
[5] Kong et al. (2003) Abstract from 11th International Symposium on Viral Hepatitis and Liver Disease, 6-10 Apr. 2003, Sydney Australia.
[6] Boland et al. (2003) Abstract from 11th International Symposium on Viral Hepatitis and Liver Disease, 6-10 Apr. 2003, Sydney Australia.
[7] Starkel et al. (2003) Abstract 61 from 55th Annual Meeting of the American Association for the Study of Liver Diseases, 29th October to 2nd November, Boston Mass.
[8] Jacques et al. (2002) Vaccine 20:3644-9.
[9] Tong et al. (2005) Kidney International 68:2298-303.
[10] Boland et al. (2004) Vaccine 23:316-20.
[11] WO93/19780.
[12] WO96/26741.
[13] U.S. Pat. No. 5,972,346.
[14] U.S. Pat. No. 6,488,934.
[15] UK patent application GB-A-2220211.
[16] Myers et al. (1990) pages 145-156 of Cellular and molecular aspects of endotoxin reactions.
[17] Ulrich (2000) Chapter 16 (pages 273-282) of reference 1.
[18] Johnson et al. (1999) J Med Chem 42:4640-9.
[19] Baldrick et al. (2002) Regulatory Toxicol Pharmacol 35:398-413.
[20] WO 94/21292.
[21] U.S. Pat. No. 6,013,264.
[22] U.S. Pat. No. 4,624,918.
[23] WO03/066094.
[24] Harper et al. (2004) Lancet 364(9447): 1757-65.
[25] EP-A-0139417.
[26] U.S. Pat. No. 5,928,902.
[27] WO 90/01496.
[28] Nony et al. (2001) Vaccine 27:3645-51.

1. An adjuvant composition comprising: (i) an aluminum phosphate adjuvant; and (ii) a 3-O-deacylated monophosphoryl lipid A adjuvant, characterised in that at least 50% of the 3-O-deacylated monophosphoryl lipid A is adsorbed to the aluminum phosphate adjuvant.

2. The adjuvant composition of claim 1, wherein the composition has less than 5 μg/ml of unadsorbed 3-O-deacylated monophosphoryl lipid A.

3. The adjuvant composition of claim 1 or claim 2, wherein at least 95% of the 3-O-deacylated monophosphoryl lipid A is adsorbed to the aluminum phosphate adjuvant.

4. The adjuvant composition of claim 1, wherein the 3-O-deacylated monophosphoryl lipid A adjuvant comprises a mixture of acylated disaccharides, wherein each disaccharide: (a) has two β-1′,6-linked 2-deoxy-2-aminoglucose monosaccharide subunits; (b) is phosphorylated at the 4′ position; (c) is unsubstituted at the 1, 3 and 6′ positions, (d) is O-acylated at the 3′ position, and (e) is N-acylated at the 2 and 2′ positions, and wherein the mixture of acylated disaccharides includes at least 10% by weight of a component in which each of the acyl groups at the 2, 2′ and 3′ positions is itself substituted at an aliphatic carbon atom with an O-acyl group.

5. The adjuvant composition of claim 1 further comprising a triethylammonium ion.

6. The adjuvant composition of claim 1, wherein the composition has an osmolality between 200 and 400 mOsm/kg.

7. The adjuvant composition of claim 1, wherein the composition has a pH of between 5 and 7.5.

8. An immunogenic composition comprising: (i) an aluminum phosphate adjuvant; (ii) a 3-O-deacylated monophosphoryl lipid A adjuvant; and (iii) an antigen, characterised in that at least 50% of the 3-O-deacylated monophosphoryl lipid A is adsorbed to the aluminum phosphate adjuvant.

9. The composition of claim 1 of claim 8, wherein the aluminium phosphate adjuvant is amorphous.

10. The composition of claim 9, wherein the antigen is a hepatitis B virus surface antigen (HBsAg).

11. The composition of claim 10, wherein at least 50% of the HBsAg (preferably at least 90%) is adsorbed to the aluminum phosphate adjuvant.

12. The composition of claim 10 or claim 11, wherein the antigen is yeast-expressed HBsAg in the form of substantially-spherical particles including a lipid matrix comprising phospholipids.

13. The composition of claim 12, wherein the yeast is Saccharomyces cerevisiae.

14. The composition of claim 10 or claim 11, wherein a 0.5 ml dose of the composition has: about 50 μg 3-O-deacylated monophosphoryl lipid A; about 0.5 mg aluminum phosphate (expressed in terms of Al³⁺); and about 20 μg/ml HBsAg.

15. The composition of claim 8 or claim 9, wherein the antigen is a mixed particle (RTS,S) expressed in yeast, comprising: (a) RTS, which is a hybrid protein comprising substantially all the C-terminal portion of P. falciparum CS protein linked via four amino acids of the preS2 portion of HBV surface antigen to HBsAg; and (b) S, which is a hepatitis B virus surface antigen.

16. The composition of any one of claim 8, packaged into a syringe.

17. The composition of claim 16, wherein the syringe is made from a borosilicate glass and has a tip cap made of butyl rubber.

18. A process for preparing the composition of claim 8, comprising the steps of: (a) mixing the antigen and the aluminum phosphate adjuvant; and then (b) combining the 3-O-deacylated monophosphoryl lipid A adjuvant with the antigen/aluminum phosphate mixture.

19. The process of claim 18, wherein the antigen adsorbs to the aluminum phosphate adjuvant in step (a).

20. The process of claim 18 or claim 19, further comprising, after step (b), a step of extracting and packaging a 0.5 ml sample of the mixture into a container.

21. The process of claim 20, wherein the container is a glass syringe.

22. A method for raising an immune response in a patient comprising administering to the patient a composition comprising: (i) an antigen; (ii) an aluminum phosphate adjuvant; and (iii) a 3-O-deacylated monophosphoryl lipid A adjuvant, wherein at least 50% of the 3-O-deacylated monophosphoryl lipid A is adsorbed to the aluminum phosphate adjuvant.

23. A method of claim 22, wherein the composition is administered via intramuscular injection.

24. A method of claim 22 or claim 23, wherein the antigen is RBsAg.

25. A method of claim 24, wherein the composition is administered by an immunisation schedule with doses at 0, 1, 2 & 6 months, where time 0 is the first dose.

26. The method of claim 24, wherein the patient is a hemodialysis adult.
